(19) United States
(12) Patent Application Publication
(10) Pub. No.: US 2020/0338245 A1
(43) Pub. Date: Oct. 29, 2020

(54) NEGATIVE PRESSURE WOUND THERAPY CANISTER

(71) Applicant: KCI LICENSING, INC., San Antonio, TX (US)

(72) Inventors: Benjamin A. PRATT, Poole (GB); James K. SEDDON, Wimborne (GB); Christopher B. LOCKE, Bournemouth (GB)

(73) Assignee: KCI LICENSING, INC., San Antonio, TX (US)

(21) Appl. No.: 16/758,271

(22) PCT Filed: Oct. 23, 2018
(86) PCT No.: PCT/US2018/057044

§ 371 Date: Apr. 22, 2020

Related U.S. Application Data
(60) Provisional application No. 62/577,544, filed on Oct. 26, 2017.

(51) Int. Cl. A61M 1/00

(52) U.S. Cl. CPC A61M 1/0088; A61M 2205/75; A61M 1/005; A61M 1/0001

(57) ABSTRACT

A canister for a negative pressure wound therapy device includes a receptacle, a lid, and a port cover. The receptacle is configured to contain wound exudate collected from a wound site. The lid is configured to attach to the receptacle and includes a port extending through the lid. The port cover is pivotally attached to the lid and configured to pivot between a closed position in which the port cover covers the port and an open position in which the port cover uncovers the port. The port cover includes a buoyant float configured to float in the wound exudate and a non-buoyant mass configured to sink in the wound exudate. The buoyant float and the non-buoyant mass cause the port cover to pivot between the closed position and the open position responsive to a level of the wound exudate within the receptacle and/or an orientation of the receptacle.

### CROSS-REFERENCE TO RELATED APPLICATIONS

This application claims the benefit of priority to U.S. Provisional Application No. 62/577,544, filed on Oct. 26, 2017, which is incorporated herein by reference in its entirety.

### BACKGROUND

The present disclosure relates generally to wound therapy systems and devices, and more particularly to a canister for a negative pressure wound therapy device.

Negative pressure wound therapy (NPWT) is a type of wound therapy that involves applying negative pressure (relative to atmospheric pressure) to a wound site to promote wound healing. Some NPWT systems include a pump which operates to maintain the wound site at negative pressure by removing wound exudate from the wound site. The wound exudate is typically routed to a canister or other container fluidly connected to the pump where the wound exudate is stored until emptied by a user.

### SUMMARY

One implementation of the present disclosure is a canister for a negative pressure wound therapy device. The canister includes a receptacle, a lid, and a port cover. The receptacle is configured to contain wound exudate collected from a wound site. The lid is configured to attach to the receptacle and includes a port extending through the lid. The port cover is pivotally attached to the lid and configured to pivot between a closed position in which the port cover covers the port and an open position in which the port cover uncovers the port. The port cover includes a buoyant float configured to float in the wound exudate and a non-buoyant mass configured to sink in the wound exudate. The buoyant float and the non-buoyant mass cause the port cover to pivot between the closed position and the open position responsive to at least one of a level of the wound exudate within the receptacle or an orientation of the receptacle.

In some embodiments, the port is configured to fluidly couple the receptacle to a therapy device to allow the therapy device to pump air out of the receptacle through the port. In some embodiments, the canister includes a filter covering the port. The port cover can be configured to prevent the wound exudate from contacting the filter when the port cover is in the closed position.

In some embodiments, the lid includes a lower surface facing toward an inside of the receptacle when the lid is attached to the receptacle. In some embodiments, the port cover is suspended from the lower surface of the lid such that the port cover is located within the receptacle when the lid is attached to the receptacle.

In some embodiments, the port cover is configured to pivot about a first axis and the buoyant float is offset from the first axis such that the buoyant float causes the port cover to pivot about the first axis responsive to the level of the wound exudate within the receptacle.

In some embodiments, the buoyant float produces a first moment about the first axis in a first direction of rotation when the level of the wound exudate within the receptacle is at or above the buoyant float. The first moment may cause the port cover to pivot toward the closed position. In some embodiments, the non-buoyant mass produces a second moment about the first axis in a second direction of rotation opposite the first direction of rotation. The second moment may cause the port cover to pivot toward the open position.

In some embodiments, the first moment is greater than the second moment when the level of the wound exudate within the receptacle is at or above the buoyant float such that port cover pivots toward the closed position when the level of the wound exudate within the receptacle is at or above the buoyant float. In some embodiments, the first moment is less than the second moment when the level of the wound exudate within the receptacle is below the buoyant float such that port cover pivots toward the open position when the level of the wound exudate within the receptacle is below the buoyant float.

In some embodiments, the non-buoyant mass is pivotally attached to the buoyant float and configured to pivot about a second axis aligned with the buoyant float. In some embodiments, the non-buoyant mass is configured to pivot about the second axis between an aligned position and a misaligned position. In the aligned position, the non-buoyant mass may be substantially horizontally aligned with the buoyant float such that both the buoyant float and the non-buoyant mass are horizontally offset from the first axis by substantially equal distances. In the misaligned position, the non-buoyant mass may be horizontally misaligned with the buoyant float such that a horizontal distance between the non-buoyant mass and the first axis exceeds a horizontal distance between the buoyant float and the first axis. In some embodiments, the non-buoyant mass is in the aligned position when the canister is in an upright orientation and the misaligned position when the canister is in an inverted orientation.

In some embodiments, the buoyant float produces a first moment about the first axis in a first direction of rotation when the canister is in an inverted position. The first moment may cause the port cover to pivot toward the open position. In some embodiments, the non-buoyant mass produces a second moment about the first axis in a second direction of rotation opposite the first direction of rotation when the canister is in the inverted position. The second moment may cause the port cover to pivot toward the closed position.

In some embodiments, the first moment is less than the second moment when the canister is in the inverted position such that port cover pivots toward the closed position when the canister is in the inverted position. In some embodiments, a fluid pressure exerted by the fluid within the receptacle produces a third moment which acts upon the port cover in the second direction of rotation in combination with the second moment to cause the port cover to pivot toward the closed position when the canister is in the inverted position. In some embodiments, a sum of the second moment and the third moment exceeds the first moment when the canister is in the inverted position such that port cover pivots toward the closed position when the canister is in the inverted position.

In some embodiments, the non-buoyant mass acts at a first distance offset from the first axis to produce the second moment when the canister is in an upright position and acts at a second distance greater than the first distance offset from the first axis to produce the second moment when the canister is in an inverted orientation.

In some embodiments, the non-buoyant mass is suspended from the buoyant float such that the non-buoyant mass and the buoyant float are horizontally aligned when the canister is in an upright orientation and horizontally misaligned when the canister is in an inverted orientation. In some embodiments, the non-buoyant mass is configured to pivot about a second axis aligned with the buoyant float such that the non-buoyant mass is horizontally offset from the buoyant float when the canister is in an inverted orientation.

Another implementation of the present disclosure is a canister including a receptacle, a lid, and port cover. The receptacle is configured to contain a fluid. The lid is configured to attach to the receptacle and includes a port extending through the lid. The port cover is pivotally attached to the lid and configured to pivot between a closed position in which the port cover covers the port and an open position in which the port cover uncovers the port. The port cover includes a buoyant float configured to float in the fluid and a non-buoyant mass configured to sink in the fluid. The buoyant float and the non-buoyant mass cause the port cover to pivot between the closed position and the open position responsive to at least one of a level of the fluid within the receptacle or an orientation of the receptacle.

In some embodiments, the lid includes a lower surface facing toward an inside of the receptacle when the lid is attached to the receptacle. In some embodiments, the port cover is suspended from the lower surface of the lid such that the port cover is located within the receptacle when the lid is attached to the receptacle.

In some embodiments, the port cover is configured to pivot about a first axis and the buoyant float is offset from the first axis such that the buoyant float causes the port cover to pivot about the first axis responsive to the level of the fluid within the receptacle.

In some embodiments, the buoyant float produces a first moment about the first axis in a first direction of rotation when the level of the fluid within the receptacle is at or above the buoyant float. The first moment may cause the port cover to pivot toward the closed position. In some embodiments, the non-buoyant mass produces a second moment about the first axis in a second direction of rotation opposite the first direction of rotation. The second moment may cause the port cover to pivot toward the open position.

In some embodiments, the first moment is greater than the second moment when the level of the fluid within the receptacle is at or above the buoyant float such that port cover pivots toward the closed position when the level of the fluid within the receptacle is at or above the buoyant float. In some embodiments, the first moment is less than the second moment when the level of the fluid within the receptacle is below the buoyant float such that port cover pivots toward the open position when the level of the fluid within the receptacle is below the buoyant float.

In some embodiments, the non-buoyant mass is pivotally attached to the buoyant float and configured to pivot about a second axis aligned with the buoyant float. In some embodiments, the non-buoyant mass is configured to pivot about the second axis between an aligned position and a misaligned position. In the aligned position, the non-buoyant mass may be substantially horizontally aligned with the buoyant float such that both the buoyant float and the non-buoyant mass are horizontally offset from the first axis by substantially equal distances. In the misaligned position, the non-buoyant mass may be horizontally misaligned with the buoyant float such that a horizontal distance between the non-buoyant mass and the first axis exceeds a horizontal distance between the buoyant float and the first axis. In some embodiments, the non-buoyant mass is in the aligned position when the canister is in an upright orientation and is in the misaligned position when the canister is in an inverted orientation.

In some embodiments, the buoyant float produces a first moment about the first axis in a first direction of rotation when the canister is in an inverted position. The first moment may cause the port cover to pivot toward the open position. In some embodiments, the non-buoyant mass produces a second moment about the first axis in a second direction of rotation opposite the first direction of rotation when the canister is in the inverted position. The second moment may cause the port cover to pivot toward the closed position.

In some embodiments, the first moment is less than the second moment when the canister is in the inverted position such that port cover pivots toward the closed position when the canister is in the inverted position. In some embodiments, a fluid pressure exerted by the fluid within the receptacle produces a third moment which acts upon the port cover in the second direction of rotation in combination with the second moment to cause the port cover to pivot toward the closed position when the canister is in the inverted position. In some embodiments, a sum of the second moment and the third moment exceeds the first moment when the canister is in the inverted position such that port cover pivots toward the closed position when the canister is in the inverted position.

In some embodiments, the non-buoyant mass acts at a first distance offset from the first axis to produce the second moment when the canister is in an upright position and acts at a second distance greater than the first distance offset from the first axis to produce the second moment when the canister is in an inverted orientation.

In some embodiments, the non-buoyant mass is suspended from the buoyant float such that the non-buoyant mass and the buoyant float are horizontally aligned when the canister is in an upright orientation and horizontally misaligned when the canister is in an inverted orientation. In some embodiments, the non-buoyant mass is configured to pivot about a second axis aligned with the buoyant float such that the non-buoyant mass is horizontally offset from the buoyant float when the canister is in an inverted orientation.

Another implementation of the present disclosure is a canister including a receptacle, a lid, a cover plate, one or more buoyant floats, and one or more non-buoyant masses. The receptacle is configured to contain a fluid. The lid is configured to attach to the receptacle and includes a port extending through the lid. The cover plate is attached to the lid and configured to move between a closed position in which the port cover covers the port and an open position in which the port cover uncovers the port. The one or more buoyant floats are configured to float in the fluid, whereas the one or more non-buoyant masses configured to sink in the fluid. The buoyant floats and the non-buoyant masses cause the cover plate to move between the closed position and the open position responsive to at least one of a level of the fluid within the receptacle or an orientation of the receptacle.

In some embodiments, the lid includes a lower surface facing toward an inside of the receptacle when the lid is attached to the receptacle. In some embodiments, the buoyant floats and non-buoyant masses are suspended from the lower surface of the lid and located within the receptacle when the lid is attached to the receptacle.

In some embodiments, the cover plate is configured to translate linearly toward the lid to move into the closed position and linearly away from the lid to move into the open position. In some embodiments, the buoyant floats and non-buoyant masses are configured to engage the cover plate to cause the cover plate to move between the open position and the closed position responsive to the level of the fluid within the receptacle.

In some embodiments, the buoyant floats apply a first force to the cover plate in a first direction when the level of the fluid within the receptacle is at or above the buoyant floats. The first force may cause the cover plate to move toward the closed position. In some embodiments, the non-buoyant masses apply a second force to the cover plate in a second direction opposite the first direction. The second force may cause the cover plate to move toward the open position.

In some embodiments, the first force is greater than the second force when the level of the fluid within the receptacle is at or above the buoyant floats such that cover plate moves toward the closed position when the level of the fluid within the receptacle is at or above the buoyant floats. In some embodiments, the first force is less than the second force when the level of the fluid within the receptacle is below the buoyant floats such that cover plate moves toward the open position when the level of the fluid within the receptacle is below the buoyant floats.

In some embodiments, the buoyant floats apply a first force to the cover plate in a first direction when the canister is in an inverted position. The first force may cause the cover plate to pivot move the open position. In some embodiments, the non-buoyant masses apply a second force to the cover plate in a second direction opposite the first direction when the canister is in the inverted position. The second force may cause the cover plate to move toward the closed position. In some embodiments, the first force is less than the second force when the canister is in the inverted position such that cover plate moves toward the closed position when the canister is in the inverted position.

In some embodiments, a fluid pressure exerted by the fluid within the receptacle applies a third force to the cover plate in the second direction in combination with the second force to cause the cover plate to move toward the closed position when the canister is in the inverted position. In some embodiments, a sum of the second force and the third force exceeds the first force when the canister is in the inverted position such that cover plate moves toward the closed position when the canister is in the inverted position.

In some embodiments, the buoyant floats include a plurality of floating levers and the non-buoyant masses comprise a plurality of sinking levers. In some embodiments, each of the floating levers is paired with one of the sinking levers and located on an opposite side of the cover plate relative to the sinking lever with which the floating lever is paired. In some embodiments, each pair comprising one of the floating levers and one of the sinking levers is configured to pivot within a shared plane corresponding to the pair.

In some embodiments, the buoyant floats and the non-buoyant masses are arranged in an alternating sequence along a perimeter of the cover plate. In some embodiments, the buoyant floats and the non-buoyant masses are arranged along a perimeter of the cover plate such that each of the buoyant floats is located opposite one of the non-buoyant masses.

Another implementation of the present disclosure is a canister for a negative pressure wound therapy device. The canister includes a receptacle configured to contain wound exudate collected from a wound site and a lid configured to attach to the receptacle. The lid includes a first surface, a first port extending through the first surface, a second surface substantially parallel to the first surface, and a second port extending through the second surface. The second port is offset from the first port. The lid further includes an intermediate layer between the first surface and the second surface and a pneumatic pathway extending through the intermediate layer in a direction substantially parallel to the first surface and the second surface. The pneumatic pathway connects the first port and the second port.

In some embodiments, the first surface faces away from the receptacle when the lid is attached to the receptacle and the second surface faces toward the receptacle when the lid is attached to the receptacle.

In some embodiments, the canister includes a filter covering the first port. In some embodiments, the canister includes comprising an absorbent capsule located within the pneumatic pathway and covering the second port.

In some embodiments, the first port is configured to fluidly couple the receptacle to a pump of the negative pressure wound therapy device to allow the pump to draw a vacuum within the receptacle by pumping air out of the receptacle via the first port.

In some embodiments, the canister includes a third port extending through the first surface, the intermediate layer, and the second surface in a direction substantially perpendicular to the first surface and the second surface. In some embodiments, the third port is configured to fluidly couple the receptacle to the wound site to allow the wound exudate to enter the receptacle via the third port.

In some embodiments, the first port is located proximate a perimeter of the lid and the second port is located proximate a midpoint of the lid. In other embodiments, the first port is located proximate a midpoint of the lid and the second port is located proximate a perimeter of the lid.

In some embodiments, the pneumatic pathway is a helical pathway within the intermediate layer. In some embodiments, the pneumatic pathway is a groove in the first surface and extends into the intermediate layer from the first surface.

In some embodiments, the pneumatic pathway has a lower surface that slopes downward toward the second port to guide any wound exudate within the pneumatic pathway toward the second port and into the receptacle. In some embodiments, the second surface forms a lower surface of the pneumatic pathway.

In some embodiments, the canister includes one or more one-way valves extending through the second surface and into the pneumatic pathway. In some embodiments, the one-way valves are configured to allow fluid flow from the pneumatic pathway into the receptacle through the one-way valves and prevent fluid flow from the receptacle into pneumatic pathway through the one-way valves.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 is a drawing of a negative pressure wound therapy (NPWT) system including a NPWT device fluidly connected with a wound site, according to an exemplary embodiment.

FIG. 2 is a cross-sectional view of a canister for a NPWT device, showing a buoyant float and a non-buoyant mass configured to move a port cover between an open position and a closed position, according to an exemplary embodiment.

FIG. 3 is another cross-sectional view of the canister of FIG. 2, showing the port cover in a closed position, according to an exemplary embodiment.

FIG. 4 is another cross-sectional view of the canister of FIG. 2, showing the canister inverted and the port cover in the closed position, according to an exemplary embodiment.

FIG. 5 is a cross-sectional view of another canister for a NPWT device, showing a plurality of floating and sinking levers configured to move a cover plate between an open position and a closed position, according to an exemplary embodiment.

FIG. 6 is a top view of the canister of FIG. 5, showing the plurality of floating and sinking levers arranged around a perimeter of the cover plate, according to an exemplary embodiment.

FIG. 7 is another cross-sectional view of the canister of FIG. 5, showing the operation of the floating and sinking levers when the level of fluid in the canister is below the floating and sinking levers, according to an exemplary embodiment.

FIG. 8 is a detail view of the cross-sectional view shown in FIG. 7, according to an exemplary embodiment.

FIG. 9 is another cross-sectional view of the canister of FIG. 5, showing the operation of the floating and sinking levers when the level of fluid in the canister is at the level of the floating levers, according to an exemplary embodiment.

FIG. 10 is a detail view of the cross-sectional view shown in FIG. 9, according to an exemplary embodiment.

FIG. 11 is another cross-sectional view of the canister of FIG. 5, showing the operation of the floating and sinking levers as the level of fluid increases above the level of the sinking levers, according to an exemplary embodiment.

FIG. 12 is a detail view of the cross-sectional view shown in FIG. 11, according to an exemplary embodiment.

FIG. 13 is another cross-sectional view of the canister of FIG. 5, showing the operation of the floating and sinking levers when the level of fluid in the canister is at the top of the canister, according to an exemplary embodiment.

FIG. 14 is a detail view of the cross-sectional view shown in FIG. 13, according to an exemplary embodiment.

FIG. 15 is another cross-sectional view of the canister of FIG. 5, showing the operation of the floating and sinking levers when the canister is inverted, according to an exemplary embodiment.

FIG. 16 is another cross-sectional view of the canister of FIG. 5, showing the operation of the floating and sinking levers when the canister is oriented horizontally, according to an exemplary embodiment.

FIG. 17A is an exploded view of another canister for a NPWT device, showing a complex pneumatic pathway in a lid of the canister, according to an exemplary embodiment.

FIG. 17B is an exploded view of the canister of FIG. 17A, showing an alternative arrangement of ports in the lid, according to an exemplary embodiment.

FIG. 18 is a top view of the canister of FIG. 17A, according to an exemplary embodiment.

FIG. 19 is a cross-sectional view of the canister of FIG. 17A, according to an exemplary embodiment.

FIG. 20 is a detail view of a portion of the cross-sectional view of FIG. 19, according to an exemplary embodiment.

FIG. 21 is another detail view of a portion of the cross-sectional view of FIG. 19, according to an exemplary embodiment.

### DETAILED DESCRIPTION

### Overview

Referring generally to the FIGURES, a canister for a negative pressure wound therapy (NPWT) device and components thereof are shown, according to various exemplary embodiments. In some embodiments, the canister includes a receptacle, a lid, and a port cover. The receptacle can be configured to contain wound exudate collected from a wound site. The lid can be configured to attach to the receptacle and may include a port extending through the lid. The port cover can be pivotally attached to the lid and configured to pivot between a closed position in which the port cover covers the port and an open position in which the port cover uncovers the port. The port cover may include a buoyant float configured to float in the wound exudate and a non-buoyant mass configured to sink in the wound exudate. The buoyant float and the non-buoyant mass may cause the port cover to pivot between the closed position and the open position responsive to at least one of a level of the wound exudate within the receptacle or an orientation of the receptacle

In some embodiments, the canister includes a receptacle, a lid, a cover plate, one or more buoyant floats, and one or more non-buoyant masses. The receptacle can be configured to contain a fluid. The lid can be configured to attach to the receptacle and may include a port extending through the lid. The cover plate can be attached to the lid and configured to move between a closed position in which the port cover covers the port and an open position in which the port cover uncovers the port. The one or more buoyant floats can be configured to float in the fluid, whereas the one or more non-buoyant masses configured to sink in the fluid. The buoyant floats and the non-buoyant masses may cause the cover plate to move between the closed position and the open position responsive to at least one of a level of the fluid within the receptacle or an orientation of the receptacle.

In some embodiments, the canister includes a receptacle configured to contain wound exudate collected from a wound site and a lid configured to attach to the receptacle. The lid may include a first surface, a first port extending through the first surface, a second surface substantially parallel to the first surface, and a second port extending through the second surface. The second port can be offset from the first port. The lid further may include an intermediate layer between the first surface and the second surface and a pneumatic pathway extending through the intermediate layer in a direction substantially parallel to the first surface and the second surface. The pneumatic pathway can connect the first port and the second port. These and other features and advantages of the canister are described in detail below.

### Negative Pressure Wound Therapy System

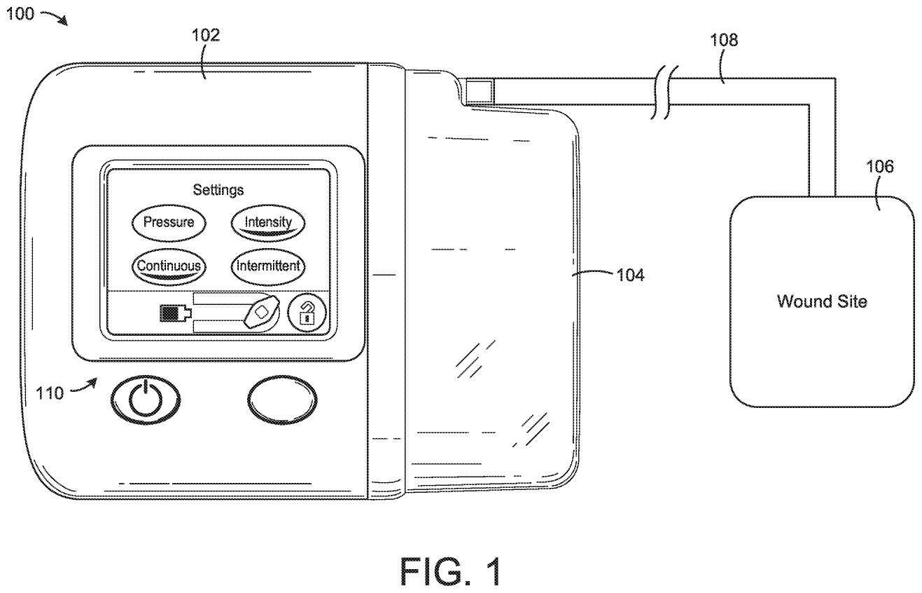

Referring now to FIG. 1, a negative pressure wound therapy (NPWT) system 100 is shown, according to an exemplary embodiment. NPWT system 100 is shown to include a therapy device 102 fluidly connected to a wound site 106 via tubing 108. Wound site 106 may include a tissue wound as well as a wound dressing that covers the tissue wound and adheres to a patient's skin. Several examples of wound dressings which can be used in combination with NPWT system 100 are described in detail in U.S. Pat. No. 7,651,484 granted Jan. 26, 2010, U.S. Pat. No. 8,394,081 granted Mar. 12, 2013, and U.S. patent application Ser. No. 14/087,418 filed Nov. 22, 2013. The entire disclosure of each of these patents and patent applications is incorporated by reference herein.

Therapy device 102 can be configured to provide negative pressure wound therapy by reducing the pressure at wound site 106. Therapy device 102 can draw a vacuum at wound site 106 (relative to atmospheric pressure) by removing wound exudate, air, and other fluids from wound site 106. Wound exudate may include fluid that filters from a patient's circulatory system into lesions or areas of inflammation. For example, wound exudate may include water and dissolved solutes such as blood, plasma proteins, white blood cells, platelets, and red blood cells. Other fluids removed from wound site 106 may include instillation fluid previously delivered to wound site 106. Instillation fluid can include, for example, a cleansing fluid, a prescribed fluid, a medicated fluid, an antibiotic fluid, or any other type of fluid which can be delivered to wound site 106 during wound treatment.

The fluids removed from wound site 106 pass through tubing 108 and are collected in canister 104. Canister 104 may be a component of therapy device 102 configured to collect wound exudate and other fluids removed from wound site 106. In some embodiments, canister 104 is detachable from therapy device 102 to allow canister 104 to be emptied and replaced as needed. During use, a lower portion of canister 104 may be filled with wound exudate and other fluids removed from wound site 106, whereas an upper portion of canister 104 may be filled with air. Therapy device 102 can be configured to draw a vacuum within canister 104 by pumping air out of canister 104. The reduced pressure within canister 104 can be translated to wound site 106 via tubing 108 such that wound site 106 is maintained at the same pressure as canister 104.

### Canister With Buoyant Float and Non-Buoyant Mass

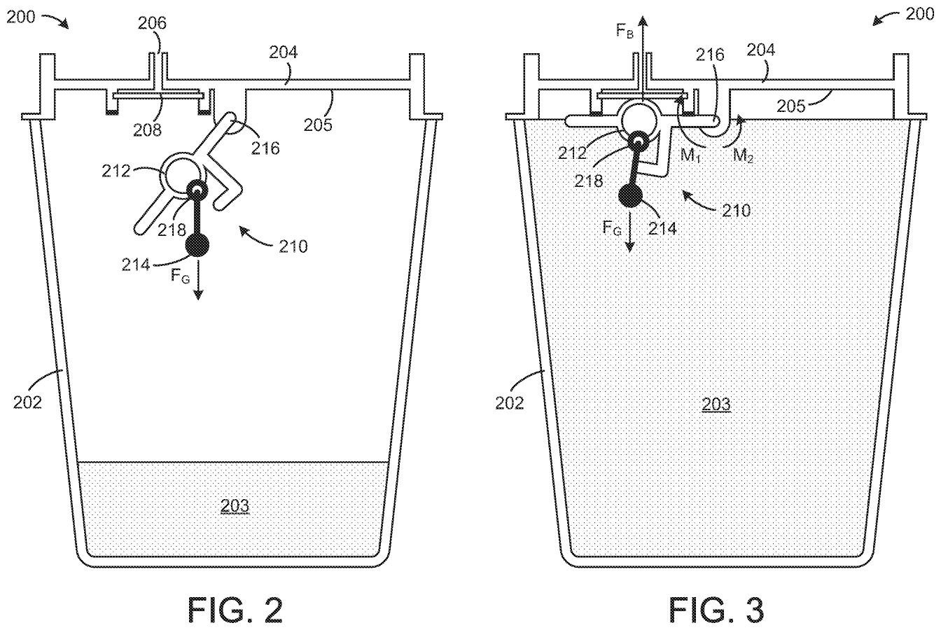

Referring now to FIGS. 2-4, a canister 200 for a negative pressure wound therapy device is shown, according to an exemplary embodiment. Canister 200 is shown to include a receptacle 202 and a lid 204. Receptacle 202 can be configured to contain a fluid 203 such as wound exudate collected from a wound site. Lid 204 can be configured to attach to receptacle 202 and may include a port 206 extending through lid 204. Port 206 is configured to fluidly couple receptacle 202 to a therapy device (e.g., therapy device 102) to allow the therapy device to pump air out of receptacle 202 through port 206. In some embodiments, canister 200 includes a filter 208 covering port 206. Filter 208 can be configured to prevent fluid 203 from exiting canister 200 via port 206 when air is pumped out of receptacle 202.

Canister 200 is shown to include a port cover 210. Port cover 210 can be pivotally attached to lid 204 and can be configured to pivot between a closed position (shown in FIG. 3) and an open position (shown in FIG. 2). In some embodiments, port cover 210 is suspended from a lower surface 205 of lid 204 (i.e., the surface which faces the inside of receptacle 202 when lid 204 is attached to receptacle 202) and pivots about an axis 216 between the closed position and open position. Port cover 210 covers port 206 when port cover 210 is in the closed position and uncovers port 206 when port cover 210 is in the open position. Port cover 210 can be configured to prevent fluid 203 within receptacle 202 from contacting filter 208 when port cover 210 is in the closed position.

Port cover 210 is shown to include a buoyant float 212 and a non-buoyant mass 214. Buoyant float 212 can be configured to float in the fluid 203 within receptacle 202, whereas non-buoyant mass 214 can be configured to sink in the fluid 203 within receptacle 202. Advantageously, buoyant float 212 and non-buoyant mass 214 cause port cover 210 to pivot between the closed position and the open position responsive to a level of the fluid 203 within receptacle 202 and/or an orientation of receptacle 202. For example, when the level of fluid 203 is below the level of buoyant float 212 and non-buoyant mass 214 (as shown in FIG. 2), both buoyant float 212 and non-buoyant mass 214 may exert a gravitational force F_{G} upon port cover 210, which causes port cover 210 to pivot into the open position. However, when the level of fluid 203 is at above the level of buoyant float 212 (as shown in FIG. 3), buoyant float 212 may exert a buoyant force F_{B} upon port cover 210. The buoyant force F_{B} may be sufficient to overcome the gravitational force F_{G} such that the net force causes port cover 210 to pivot into the closed position.

In some embodiments, buoyant float 212 is offset from axis 216. Accordingly, the buoyant force F_{B} may act at a distance from axis 216 such that the buoyant force F_{B} produces a first moment M₁ (i.e., a torque) about axis 216 in a first direction of rotation (shown as clockwise in FIG. 3) when the level of fluid 203 is at or above buoyant float 212. The first moment M₁ may cause port cover 210 to pivot toward the closed position. Similarly, non-buoyant mass 214 may produce a second moment M₂ about axis 216 in a second direction of rotation (shown as counterclockwise in FIG. 3). The second moment M₂ may cause port cover 210 to pivot toward the open position. When the level of fluid 203 within receptacle 202 is at or above buoyant float 212, the first moment M₁ may exceed the second moment M₂ such that the net moment causes port cover 210 to pivot toward the closed position. However, when the level of fluid 203 within receptacle 202 is below buoyant float 212, the first moment M₁ may be less than the second moment M₂ (or may act in the same direction as the second moment M₂) such that the net moment causes port cover 210 to pivot toward the open position.

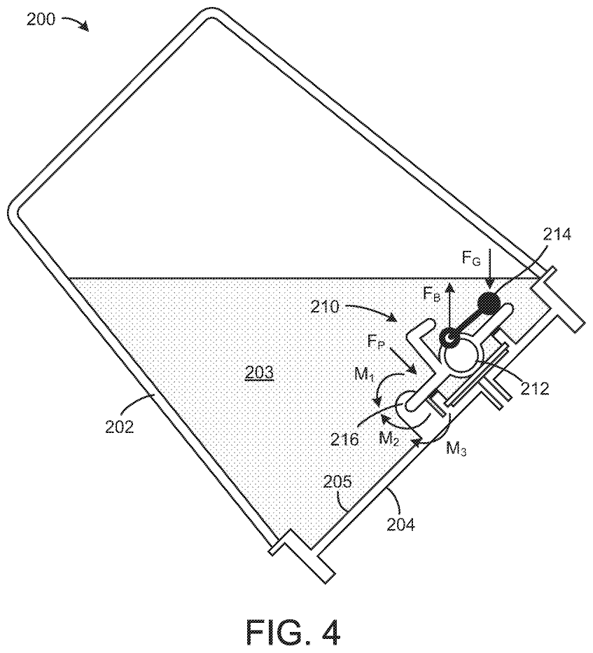

In some embodiments, non-buoyant mass 214 is pivotally coupled to buoyant float 212 and configured to pivot about a second axis 218. Axis 218 may be substantially aligned with buoyant float 212 such that non-buoyant mass 214 hangs directly below buoyant float 212. Non-buoyant mass 214 can be configured to pivot about axis 218 between an aligned position (shown in FIGS. 2-3) and a misaligned position (shown in FIG. 4). In some embodiments, non-buoyant mass 214 is in the aligned position when canister 200 is in an upright orientation, and in the misaligned position when canister 200 is in an inverted or partially inverted orientation. When non-buoyant mass 214 is in the aligned position, non-buoyant mass 214 may be substantially horizontally aligned with buoyant float 212 such that both buoyant float 212 and non-buoyant mass 214 are horizontally offset from axis 216 by substantially equal distances. However, when non-buoyant mass 214 is in the misaligned position, non-buoyant mass 214 may be horizontally misaligned with buoyant float 212 such that the horizontal distance between non-buoyant mass 214 and axis 216 exceeds the horizontal distance between buoyant float 212 and axis 216.

As shown in FIG. 4, the buoyant force F_{B} exerted by buoyant float 212 may produce a first moment M₁ about axis 216 when canister 200 is inverted. The first moment M₁ may cause port cover 210 to pivot in a first direction of rotation (counterclockwise in FIG. 4), which may cause port cover 210 to pivot into the open position. However, the gravitational force F_{G} exerted by non-buoyant mass 214 may produce a second moment M₂ about axis 216 when canister 200 is inverted. The second moment M₂ may cause port cover 210 to pivot in a second direction of rotation (clockwise in FIG. 4), which may cause port cover 210 to pivot into the closed position. In some embodiments, the second moment M₂ exceeds the first moment M₁ such that the net moment causes port cover 210 to pivot into the closed position when canister 200 is inverted.

In some embodiments, the gravitational force F_{G} exerted by non-buoyant mass 214 acts at a greater distance from axis 216 than the buoyant force F_{B} exerted by buoyant float 212 when canister 200 is in the inverted position. Accordingly, it is possible for the gravitational force F_{G} to produce a moment M₂ about axis 216 that exceeds the moment produced by the buoyant force F_{B}, even if the magnitude of the buoyant force F_{B} exceeds the magnitude of the gravitational force F_{G}. Additionally, the pressure of fluid 203 within receptacle 202 may exert a force Fₚ upon port cover 210 when canister 200 is inverted. The force Fₚ produces a third moment M₃ about axis 216 in the same direction of rotation as the moment M₂ produced by the gravitational force F_{G}. The sum of moments M₂ and M₃ may exceed moment M₁ such that the net moment causes port cover 210 to pivot into the closed position when canister 200 is inverted.

### Canister With Multiple Buoyant Floats and Non-Buoyant Masses

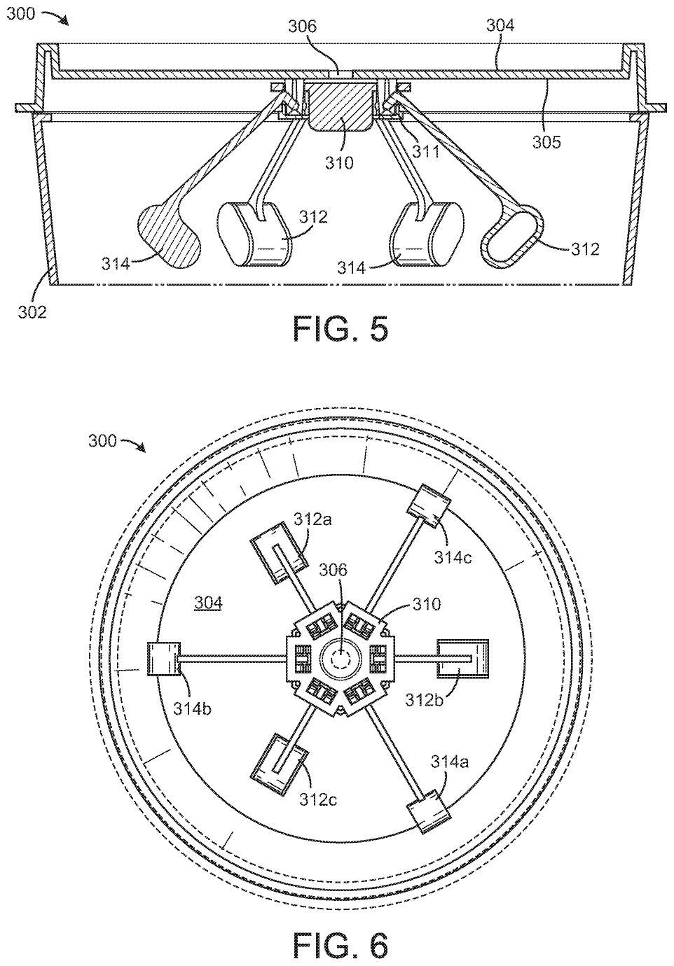

Referring now to FIGS. 5-16, another canister 300 for a negative pressure wound therapy device is shown, according to an exemplary embodiment. Canister 300 is shown to include a receptacle 302 and a lid 304. Receptacle 302 can be configured to contain a fluid 303 such as wound exudate collected from a wound site. Lid 304 can be configured to attach to receptacle 302 and may include a port 306 extending through lid 304. Port 306 is configured to fluidly couple receptacle 302 to a therapy device (e.g., therapy device 102) to allow the therapy device to pump air out of receptacle 302 through port 306. In some embodiments, canister 300 includes a filter covering port 306. The filter can be configured to prevent fluid 303 from exiting canister 300 via port 306 when air is pumped out of receptacle 302.

Canister 300 is shown to include a cover plate 310. Cover plate 310 may be attached to lid 304 and configured to move between a closed position (shown in FIG. 14) and an open position (shown in FIG. 8). In some embodiments, cover plate 310 is configured to translate linearly toward lid 304 to move into the closed position and linearly away from lid 304 to move into the open position. Cover plate 310 covers port 306 when cover plate 310 is in the closed position and uncovers port 306 when cover plate 310 is in the open position. Cover plate 310 can be configured to prevent fluid 303 within receptacle 302 from exiting receptacle via port 306 when cover plate 310 is in the closed position.

Canister 300 is shown to include a plurality of buoyant floats 312 and a plurality of non-buoyant masses 314. Buoyant floats 312 can be configured to float in the fluid 303 within receptacle 302, whereas non-buoyant masses 314 can be configured to sink in the fluid 303 within receptacle 302. In some embodiments, buoyant floats 312 include a plurality of floating levers, whereas non-buoyant masses 314 include a plurality of sinking levers. Buoyant floats 312 and non-buoyant masses 314 can be configured to pivot about a lever travel limiting ring 311. Travel limiting ring 311 may constrain the rotation of buoyant floats 312 and non-buoyant masses 314 and may cause buoyant floats 312 and non-buoyant masses 314 to engage cover plate 310 when rotated toward lid 304. For example, each of buoyant floats 312 and non-buoyant masses 314 may push cover plate 310 toward the closed position (up in FIG. 8) when rotated toward lid 304. Similarly, each of buoyant floats 312 and non-buoyant masses 314 may allow cover plate 310 to move into the open position (down in FIG. 8) when rotated away from lid 304.

In some embodiments, buoyant floats 312 and non-buoyant masses 314 are arranged in an alternating sequence along a perimeter of cover plate 310. Each of buoyant floats 312 can be paired with one of non-buoyant masses 314 and located on an opposite side of cover plate 310 relative to the non-buoyant mass 314 with which the buoyant float 312 is paired. For example, FIG. 6 shows a set of three buoyant floats 312a, 312b, and 312c, and a set of three non-buoyant masses 314a, 314b, and 314c. Buoyant float 312a may be paired with non-buoyant mass 314a and located directly opposite non-buoyant mass 314a. Similarly, buoyant float 312b may be paired with non-buoyant mass 314b and located directly opposite non-buoyant mass 314b, and buoyant float 312c may be paired with non-buoyant mass 314c and located directly opposite non-buoyant mass 314c. Each pair of a buoyant float 312 and a non-buoyant mass 314 may be configured to pivot within a shared plane corresponding to the pair.

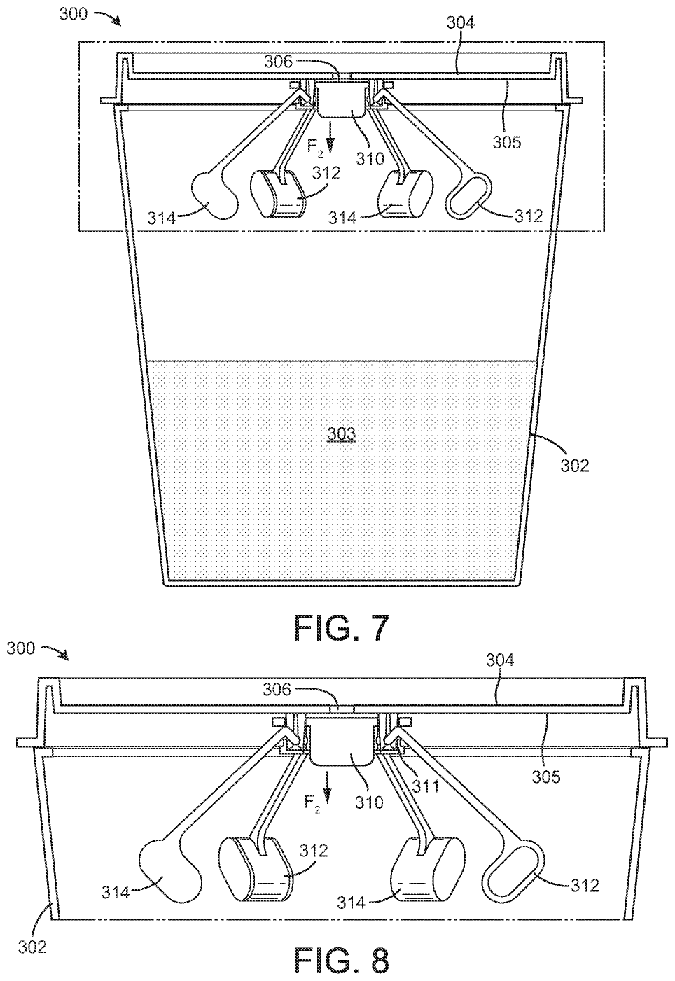

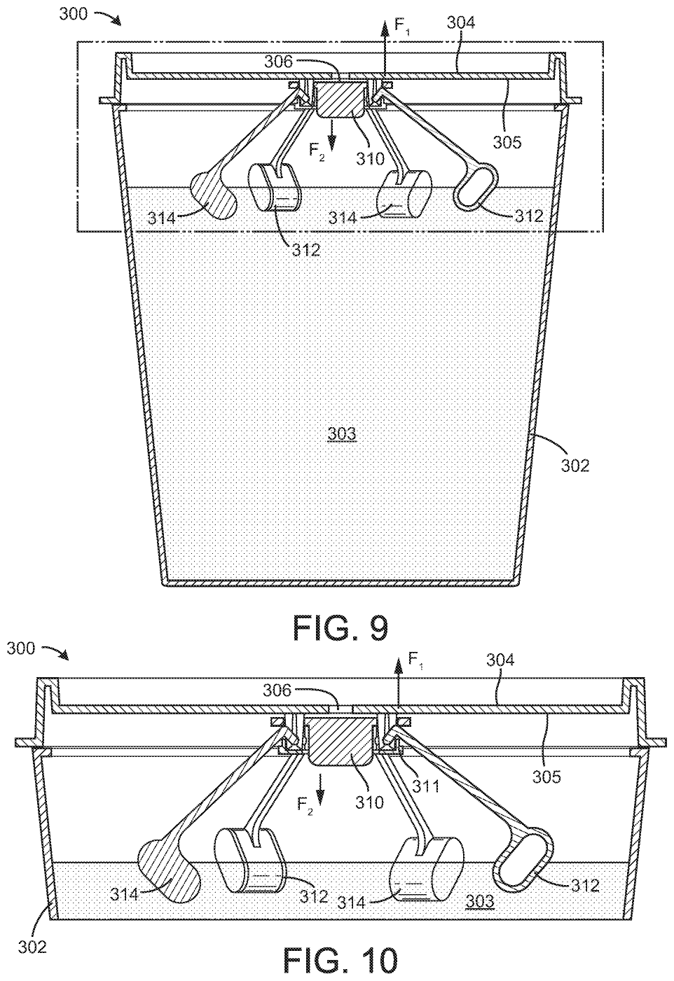

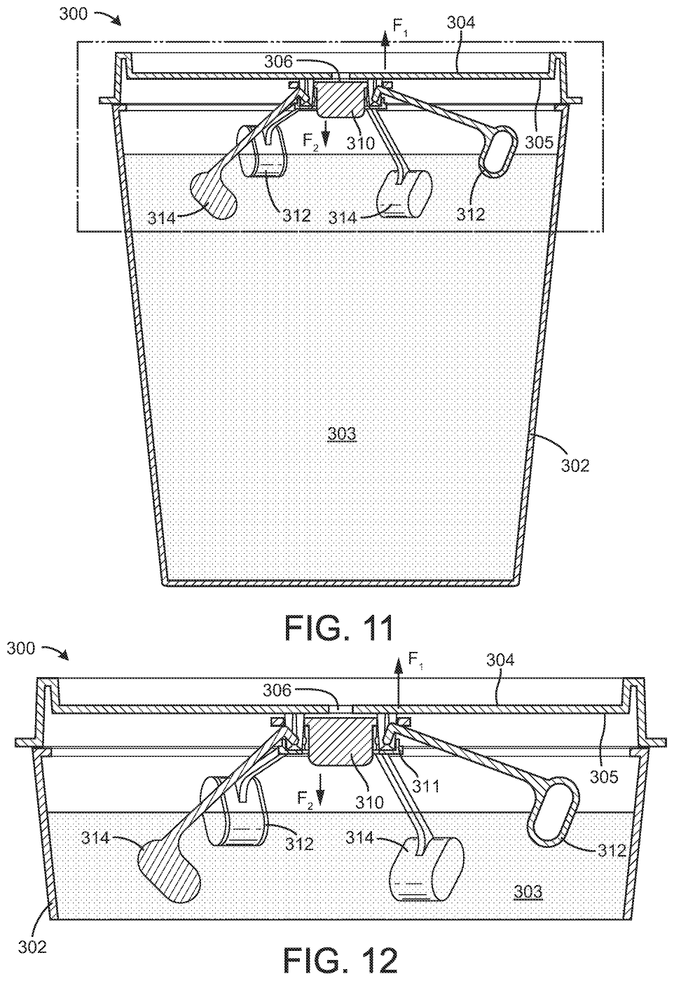

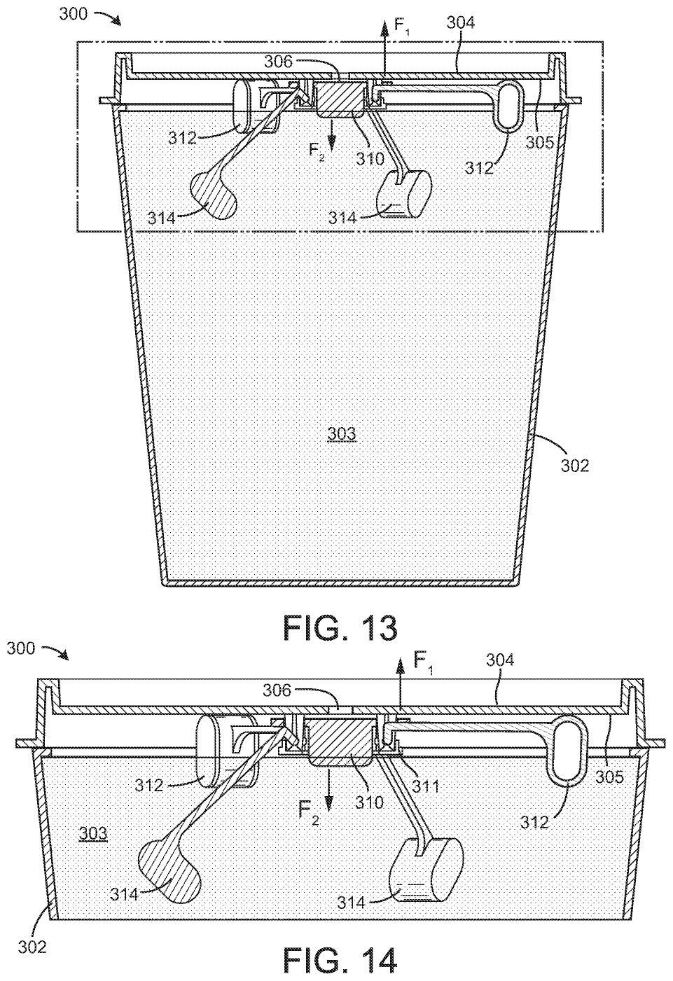

Advantageously, buoyant floats 312 and non-buoyant masses 314 cause cover plate 310 to move between the closed position and the open position responsive to a level of the fluid 303 within receptacle 302 and/or an orientation of receptacle 302. For example, when the level of fluid 303 is below the level of buoyant floats 312 and non-buoyant masses 314 (as shown in FIGS. 7-8), both buoyant floats 312 and non-buoyant masses 314 may exert a force F₂ upon cover plate 310, which causes cover plate 310 to move into the open position. However, as the level of fluid 303 increases and reaches buoyant floats 312 (as shown in FIGS. 9-10), buoyant floats 312 begin rotating toward lid 304 and exert a force F₁ upon cover plate 310. The force F₁ may be sufficient to overcome the force F₂ such that the net force causes cover plate 310 to move into the closed position. As the level of fluid 303 continues to rise, buoyant floats 312 continue to move toward lid 304 (as shown in FIGS. 11-12), which pushes cover plate 310 further toward the closed position. Buoyant floats 312 may continue to move toward cover plate 310 until buoyant floats 312 reach an end position (shown in FIGS. 13-14) at which point cover plate 310 is in the fully closed position.

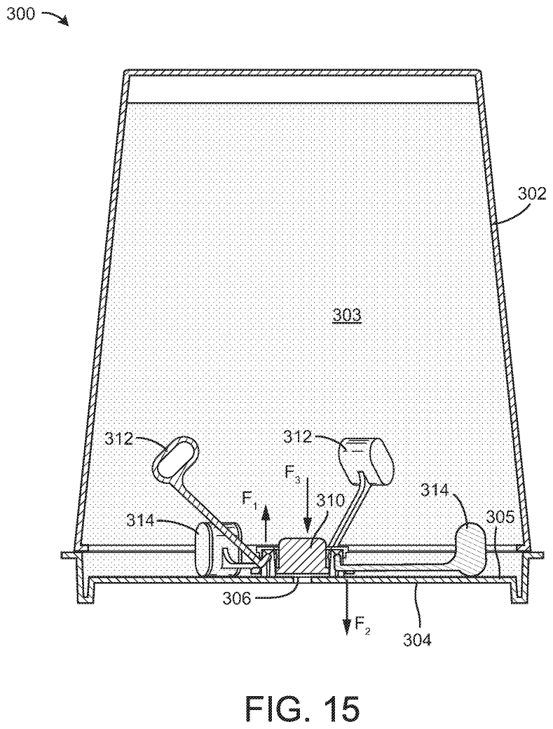

When canister 300 is inverted (as shown in FIG. 15), buoyant floats 312 float upward in fluid 303 and move away from lid 304, which causes buoyant floats 312 to exert a force F₁ upon cover plate 310. The force F₁ exerted by buoyant floats 312 may cause cover plate 310 to move toward the open position when canister 300 is inverted. However, non-buoyant masses 314 sink in fluid 303 and move toward lid 304, which causes non-buoyant masses 314 to exert a force F₂ upon cover plate 310. The force F₂ exerted by non-buoyant masses 314 may cause cover plate 310 to move toward the closed position when canister 300 is inverted. The force F₂ exerted by non-buoyant masses 314 may exceed the force F₁ exerted by buoyant floats 312 such that the net force causes cover plate 310 to move toward the closed position when canister 300 is inverted. Additionally, the pressure of fluid 303 within receptacle 302 may exert a force F₃ upon cover plate 310 when canister 200 is inverted. The force F₃ acts in the same direction as the force F₂ (i.e., downward). The sum of forces F₂ and F₃ may exceed force F₁ such that the net force causes cover plate 310 to move into the closed position when canister 300 is inverted.

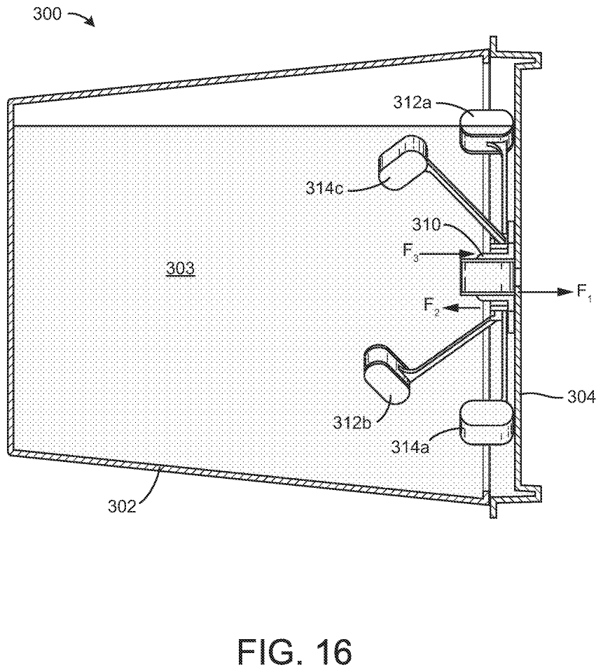

Advantageously, the arrangement of buoyant floats 312 and non-buoyant masses 314 may cause cover plate 310 to move toward the closed position when canister 300 is fully or partially inverted (i.e., not upright), regardless of the direction in which canister 300 is inverted. For example, FIG. 16 illustrates canister 300 in a horizontal orientation after being rotated approximately 90 degrees clockwise. When canister 300 is oriented horizontally, buoyant float 312a is positioned above cover plate 310, whereas buoyant float 312b is positioned below cover plate 310. Accordingly, buoyant float 312a may rotate toward cover plate 310 and may apply a closing force F₁ to cover plate 310, whereas buoyant float 312b may rotate away from cover plate 310 and may apply an opening force F₂ to cover plate 310. Conversely, non-buoyant mass 314a is positioned below cover plate 310, whereas non-buoyant mass 314c is positioned above cover plate 310. Accordingly, non-buoyant mass 314a may rotate toward cover plate 310 and may apply a closing force F₁ to cover plate 310, whereas non-buoyant mass 314c may rotate away from cover plate 310 and may apply an opening force F₂ to cover plate 310.

Due to the rotationally symmetric arrangement of buoyant floats 312 and non-buoyant masses 314 along the perimeter of cover plate 310, the closing force F₁ may always be equal to or greater than the opening force F₂, regardless of the direction in which canister 300 is rotated or inverted. For example, canister 300 could be rotated by approximately 90 degrees counterclockwise or about a different axis, which would result in a different orientation of canister 300 relative to the upright position shown in FIGS. 5-14. However, the rotational symmetry of buoyant floats 312 and non-buoyant masses 314 ensures that the forces applied by buoyant floats 312 and non-buoyant masses 314 are relatively balanced regardless of the particular direction in which canister 300 is inverted. Additionally, the force F₃ exerted by the pressure of fluid 303 within receptacle 302 acts in the same direction as the closing force F₁ when canister 300 is inverted (as shown in FIG. 16), which further pushes cover plate 310 toward the closed position.

### Canister With Complex Pneumatic Pathway

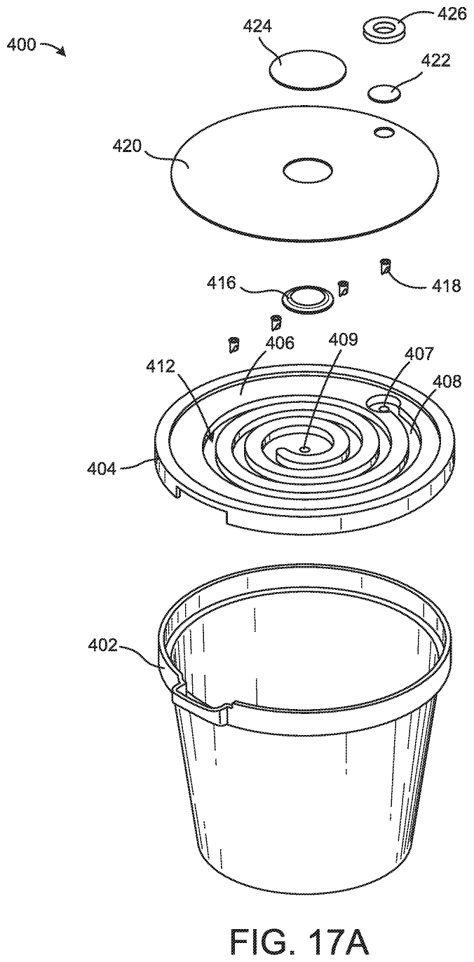

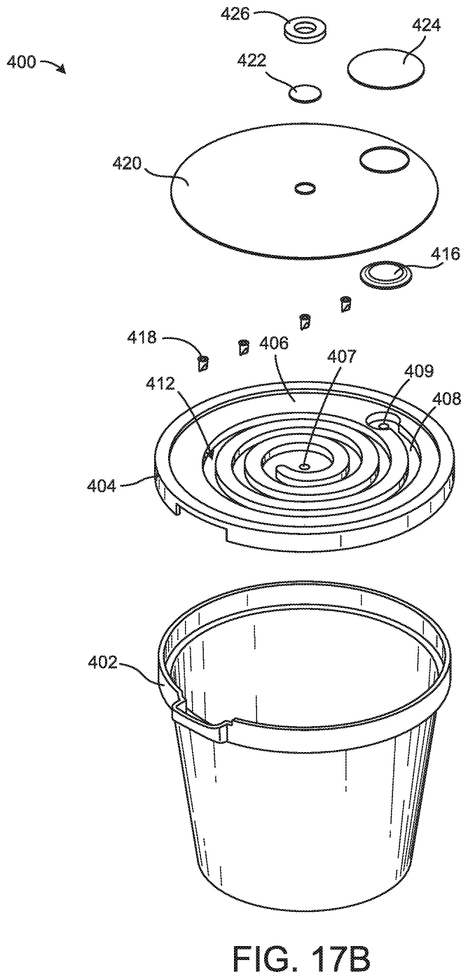

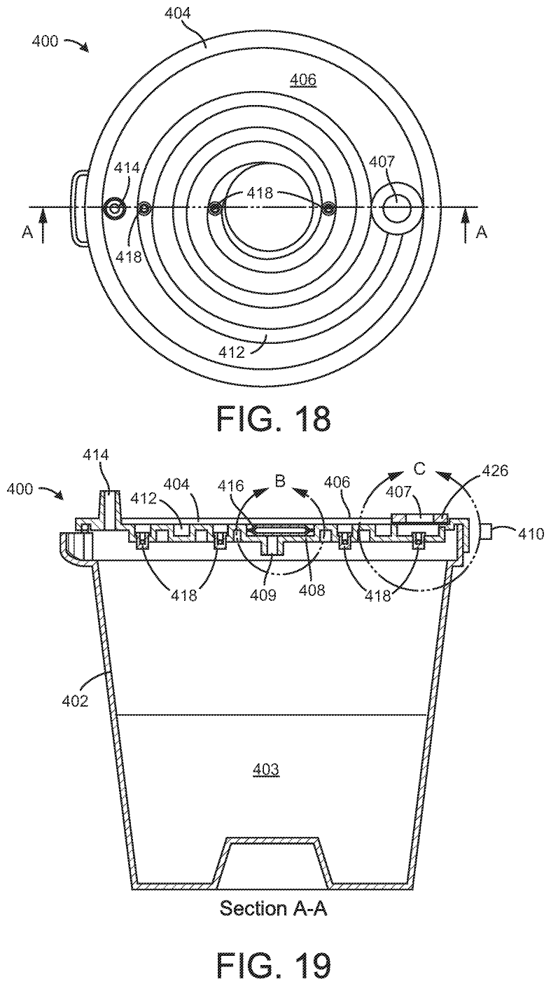

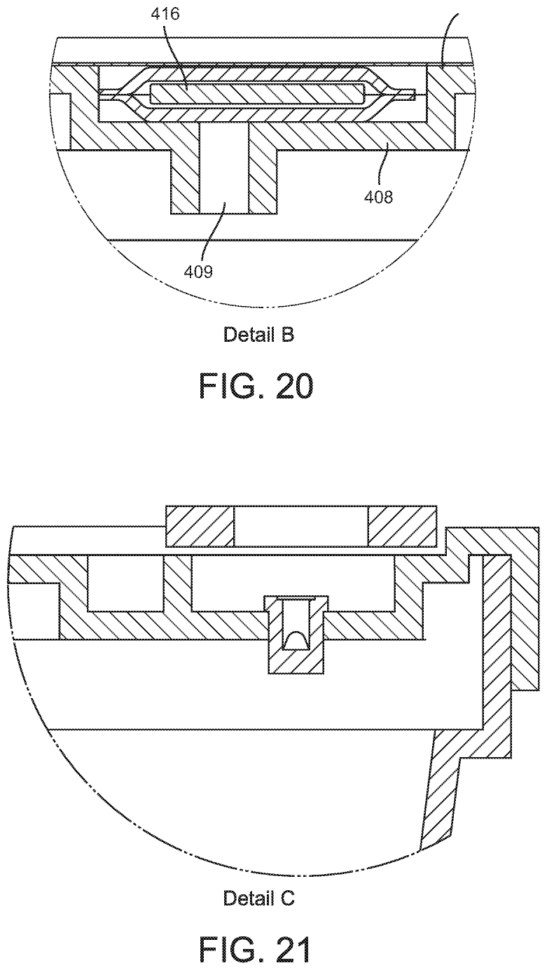

Referring now to FIGS. 17A-21, another canister 400 for a negative pressure wound therapy device is shown, according to an exemplary embodiment. Canister 400 is shown to include a receptacle 402 and a lid 404. Receptacle 402 can be configured to contain a fluid 403 such as wound exudate collected from a wound site. Lid 404 can be configured to attach to receptacle 402 and may include a complex pneumatic pathway 412 extending through lid 404. Complex pneumatic pathway 412 can be configured to baffle the flow of fluid 403 through lid 404 such that fluid 403 does not spill in the event that canister 400 is rotated or inverted.

Lid 404 is shown to include a top surface 406 and a bottom surface 408. Top surface 406 may face away from receptacle 402 when lid 404 is attached to receptacle 402, whereas bottom surface 408 may face toward receptacle 402 when lid 404 is attached to receptacle. Top surface 406 and bottom surface 408 may be substantially parallel to each other and may be separated from each other by an intermediate layer 410 located between top surface 406 and bottom surface 408. An upper port 407 extends through top surface 406 into intermediate layer 410, whereas a lower port 409 extends through bottom surface 408 into intermediate layer 410.

In some embodiments, upper port 407 is configured to fluidly couple receptacle 402 to a pump of a negative pressure wound therapy device to allow the pump to draw a vacuum within receptacle 402 by pumping air out of receptacle 402 via upper port 407. A gasket 426 can be located around upper port 407 to ensure a pneumatic seal between upper port 407 and the therapy device. In some embodiments, a filter 422 covers upper port 407, whereas an absorbent capsule 416 covers lower port 409. Absorbent capsule 416 may be located within pneumatic pathway 412. In some embodiments, a label 420 is located on top of lid 404. A drape 424 having a high moisture vapor transmission rate (MVTR) can be aligned with lower port 409 to facilitate evaporation of fluid within pneumatic pathway 412 and/or receptacle 402.

In some embodiments, upper port 407 and lower port 409 are not aligned with each other. For example, upper port 407 may be located proximate a perimeter of lid 404, whereas lower port 409 may be located proximate a midpoint of lid 404 (as shown in FIG. 17A). Alternatively, the locations of upper port 407 and lower port 409 may be swapped such that upper port 407 is located proximate the midpoint of lid 404 and lower port 409 is located proximate the perimeter of lid 404 (as shown in FIG. 17B). The embodiment shown in FIG. 17B may allow canister 400 to be emptied more easily. Upper port 407 and lower port 409 may be connected with each other by a complex pneumatic pathway 412. Pneumatic pathway 412 extends through intermediate layer 410 in a direction substantially parallel to top surface 406 and bottom surface 408 and connects upper port 407 with lower port 409 to allow airflow through lid 404.

In some embodiments, pneumatic pathway 412 has a spiral or helical shape. For example, FIG. 17A shows pneumatic pathway 412 spiraling radially inward from upper port 407 to lower port 409. In the alternative embodiment shown in FIG. 17B, pneumatic pathway 412 may spiral radially inward from lower port 409 to upper port 407. In some embodiments, pneumatic pathway 412 is shaped to maximize the length of pneumatic pathway 412 between upper port 407 and lower port 409 and/or the number of turns or bends of pneumatic pathway between upper port 407 and lower port 409. The lower surface of pneumatic pathway 412 may be sloped downward toward lower port 409 to guide any fluid within pneumatic pathway toward lower port 409 and into receptacle 402. Bottom surface 408 may form the lower surface of pneumatic pathway 412. In some embodiments, pneumatic pathway 412 is a groove in top surface 406 which extends downward into intermediate layer 410 from top surface 406.

In some embodiments, lid 404 includes one or more one-way valves 418 extending through bottom surface 408 and into pneumatic pathway 412. One-way valves 418 can be configured to allow fluid flow from pneumatic pathway 412 into receptacle 402 and prevent fluid flow from receptacle 402 into pneumatic pathway 412. This enables any fluid within pneumatic pathway 412 to drain into receptacle 402 via one-way valves 418, but prevents fluid flow in the reverse direction in the event that canister 400 is inverted. Air can be pumped out of receptacle 402 via lower port 409, pneumatic pathway 412, and upper port 407. However, the complex shape of pneumatic pathway 412 may prevent any liquid within pneumatic pathway 412 from reaching upper port 407.

In some embodiments, lid 404 includes a third port 414. Port 414 may extend through top surface 406, intermediate layer 410, and bottom surface 408 in a direction substantially perpendicular to top surface 406 and bottom surface 408. Port 414 can be configured to fluidly couple receptacle 402 to the wound site to allow wound exudate to enter receptacle 402 via port 414.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.).

For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

The present disclosure contemplates methods, systems and program products on any machine-readable media for accomplishing various operations. The embodiments of the present disclosure can be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps may differ from what is depicted. Also two or more steps can be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

1. A canister for a negative pressure wound therapy device, the canister comprising:
a receptacle configured to contain wound exudate collected from a wound site;
a lid configured to attach to the receptacle and comprising a port extending through the lid; and
a port cover pivotally attached to the lid and configured to pivot between a closed position in which the port cover covers the port and an open position in which the port cover uncovers the port, the port cover comprising:
a buoyant float configured to float in the wound exudate; and
a non-buoyant mass configured to sink in the wound exudate;
wherein the buoyant float and the non-buoyant mass cause the port cover to pivot between the closed position and the open position responsive to at least one of a level of the wound exudate within the receptacle or an orientation of the receptacle.

2. The canister of claim 1, wherein the port is configured to fluidly couple the receptacle to a therapy device to allow the therapy device to pump air out of the receptacle through the port.

3. The canister of claim 1, further comprising a filter covering the port, wherein the port cover is configured to prevent the wound exudate from contacting the filter when the port cover is in the closed position.

4. The canister of claim 1, wherein:
the lid comprises a lower surface facing toward an inside of the receptacle when the lid is attached to the receptacle; and
the port cover is suspended from the lower surface of the lid such that the port cover is located within the receptacle when the lid is attached to the receptacle.

5. The canister of claim 1, wherein:
the port cover is configured to pivot about a first axis; and
the buoyant float is offset from the first axis such that the buoyant float causes the port cover to pivot about the first axis responsive to the level of the wound exudate within the receptacle.

6. The canister of claim 5, wherein the buoyant float produces a first moment about the first axis in a first direction of rotation when the level of the wound exudate within the receptacle is at or above the buoyant float, the first moment causing the port cover to pivot toward the closed position.

7. The canister of claim 6, wherein the non-buoyant mass produces a second moment about the first axis in a second direction of rotation opposite the first direction of rotation, the second moment causing the port cover to pivot toward the open position.

8. The canister of claim 7, wherein the first moment is greater than the second moment when the level of the wound exudate within the receptacle is at or above the buoyant float such that port cover pivots toward the closed position when the level of the wound exudate within the receptacle is at or above the buoyant float.

9. The canister of claim 7, wherein the first moment is less than the second moment when the level of the wound exudate within the receptacle is below the buoyant float such that port cover pivots toward the open position when the level of the wound exudate within the receptacle is below the buoyant float.

10. The canister of claim 5, wherein the non-buoyant mass is pivotally attached to the buoyant float and configured to pivot about a second axis aligned with the buoyant float.

11. The canister of claim 10, wherein the non-buoyant mass is configured to pivot about the second axis between:
an aligned position in which the non-buoyant mass is substantially horizontally aligned with the buoyant float such that both the buoyant float and the non-buoyant mass are horizontally offset from the first axis by substantially equal distances; and
a misaligned position in which the non-buoyant mass is horizontally misaligned with the buoyant float such that a horizontal distance between the non-buoyant mass and the first axis exceeds a horizontal distance between the buoyant float and the first axis.

12. The canister of claim 11, wherein the non-buoyant mass is in:
the aligned position when the canister is in an upright orientation; and
the misaligned position when the canister is in an inverted orientation.

13. The canister of claim 5, wherein the buoyant float produces a first moment about the first axis in a first direction of rotation when the canister is in an inverted position, the first moment causing the port cover to pivot toward the open position.

14. The canister of claim 13, wherein the non-buoyant mass produces a second moment about the first axis in a second direction of rotation opposite the first direction of rotation when the canister is in the inverted position, the second moment causing the port cover to pivot toward the closed position.

15. The canister of claim 14, wherein the first moment is less than the second moment when the canister is in the inverted position such that port cover pivots toward the closed position when the canister is in the inverted position.

16. The canister of claim 14, wherein a fluid pressure exerted by the fluid within the receptacle produces a third moment which acts upon the port cover in the second direction of rotation in combination with the second moment to cause the port cover to pivot toward the closed position when the canister is in the inverted position.

17. The canister of claim 16, wherein a sum of the second moment and the third moment exceeds the first moment when the canister is in the inverted position such that port cover pivots toward the closed position when the canister is in the inverted position.

18. The canister of claim 14, wherein the non-buoyant mass:
acts at a first distance offset from the first axis to produce the second moment when the canister is in an upright position; and
acts at a second distance greater than the first distance offset from the first axis to produce the second moment when the canister is in an inverted orientation.

19. The canister of claim 1, wherein the non-buoyant mass is suspended from the buoyant float such that the non-buoyant mass and the buoyant float are:
horizontally aligned when the canister is in an upright orientation; and
horizontally misaligned when the canister is in an inverted orientation.

20. The canister of claim 19, wherein the non-buoyant mass is configured to pivot about a second axis aligned with the buoyant float such that the non-buoyant mass is horizontally offset from the buoyant float when the canister is in an inverted orientation.

21-38. (canceled)

39. A canister comprising:
a receptacle configured to contain a fluid;
a lid configured to attach to the receptacle and comprising a port extending through the lid; and
a cover plate attached to the lid and configured to move between a closed position in which the port cover covers the port and an open position in which the port cover uncovers the port;
one or more buoyant floats configured to float in the fluid; and
one or more non-buoyant masses configured to sink in the fluid;
wherein the buoyant floats and the non-buoyant masses cause the cover plate to move between the closed position and the open position responsive to at least one of a level of the fluid within the receptacle or an orientation of the receptacle.

40. The canister of claim 39, wherein:
the lid comprises a lower surface facing toward an inside of the receptacle when the lid is attached to the receptacle; and
the buoyant floats and non-buoyant masses are suspended from the lower surface of the lid and located within the receptacle when the lid is attached to the receptacle.

41. The canister of claim 39, wherein:
the cover plate is configured to translate linearly toward the lid to move into the closed position and linearly away from the lid to move into the open position; and
the buoyant floats and non-buoyant masses are configured to engage the cover plate to cause the cover plate to move between the open position and the closed position responsive to the level of the fluid within the receptacle.

42. The canister of claim 39, wherein the buoyant floats apply a first force to the cover plate in a first direction when the level of the fluid within the receptacle is at or above the buoyant floats, the first force causing the cover plate to move toward the closed position.

43-45. (canceled)

46. The canister of claim 39, wherein the buoyant floats apply a first force to the cover plate in a first direction when the canister is in an inverted position, the first force causing cover plate to pivot move the open position.

47-50. (canceled)

51. The canister of claim 39, wherein:
the buoyant floats comprise a plurality of floating levers; and
the non-buoyant masses comprise a plurality of sinking levers.

52-53. (canceled)

54. The canister of claim 39, wherein the buoyant floats and the non-buoyant masses are arranged in an alternating sequence along a perimeter of the cover plate.

55. The canister of claim 39, wherein the buoyant floats and the non-buoyant masses are arranged along a perimeter of the cover plate such that each of the buoyant floats is located opposite one of the non-buoyant masses.

56. A canister for a negative pressure wound therapy device, the canister comprising:
a receptacle configured to contain wound exudate collected from a wound site; and
a lid configured to attach to the receptacle, the lid comprising:
a first surface comprising a first port extending through the first surface;
a second surface substantially parallel to the first surface and comprising a second port extending through the second surface, the second port offset from the first port;
an intermediate layer between the first surface and the second surface; and
a pneumatic pathway extending through the intermediate layer in a direction substantially parallel to the first surface and the second surface, the pneumatic pathway connecting the first port and the second port.

57-64. (canceled)

65. The canister of claim 56, wherein the pneumatic pathway is a helical pathway within the intermediate layer.

66. The canister of claim 56, wherein the pneumatic pathway is a groove in the first surface and extends into the intermediate layer from the first surface.

67. The canister of claim 56, wherein the pneumatic pathway has a lower surface that slopes downward toward the second port to guide any wound exudate within the pneumatic pathway toward the second port and into the receptacle.

68-70. (canceled)
